(19) United States
(12) Patent Application Publication
(10) Pub. No.: US 2014/0248227 A1
(43) Pub. Date: Sep. 4, 2014

(54) DEODORIZING COMPOSITIONS

(71) Applicant: Henkel AG & Co. KGaA, Duesseldorf (DE)

(72) Inventors: Thomas Doering, Dormagen (DE); Gertraud Teckenbrock, Sprockhoevel (DE)

(73) Assignee: Henkel AG & Co. KGaA, Duesseldorf (DE)

(21) Appl. No.: 14/279,649

(22) Filed: May 16, 2014

Related U.S. Application Data
(63) Continuation of application No. PCT/EP2012/073029, filed on Nov. 20, 2012.

(30) Foreign Application Priority Data
Nov. 23, 2011 (DE) 10 2011 086923.9

(51) Int. Cl. A61K 8/49; A61Q 15/00; A61K 8/33; A61K 8/34; A61K 8/26; A61K 8/28

(52) U.S. Cl. CPC A61K 8/498; A61K 8/26; A61K 8/28; A61K 8/33; A61K 8/34; A61Q 15/00
USPC 424/66; 424/68; 512/13

(57) ABSTRACT

Cosmetic compositions for reducing body odor include a) at least one alkoxybenzene compound having the structural formula (I), having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %, b) at least one compound selected from cyclic monoterpene epoxies in a total quantity from 0.01 to 1 wt % and menthol in a total quantity from 0.09 to 5 wt %, and from mixtures of said components, c) 0 to 7 wt % water, d) a cosmetically acceptable carrier including at least one component selected from ethanol, a cosmetic oil liquid under standard conditions, and talc, as well as mixtures thereof, as well as optionally further carrier substances, adjuvants, and active agents.

### FIELD OF THE INVENTION

The present invention generally relates to cosmetic compositions that are suitable for deodorizing the body and for reducing body odor, in particular in the axillary region and/or in the region of the feet.

### BACKGROUND OF THE INVENTION

Consumers desire reliable protection from body odor. Usual deodorants can fail, however, in a context of particularly intense body odor and if the masking effect of the perfume oil is not sufficient. A need therefore existed for deodorizing compositions that effectively reduce even stronger body odor and that have a pleasant but not too strong inherent odor, so they can be combined with a larger selection of fragrances.

It is therefore desirable to provide a deodorizing composition that effectively reduces body odor, in particular in the axillary region and/or in the region of the feet. It is further desirable to provide a deodorizing composition that effectively reduces body odor, in particular in the axillary region and/or in the region of the feet, and that has a pleasant but not too strong inherent odor. The low inherent odor of the active-agent combination makes it possible to combine the latter with a larger selection of fragrances It is further desirable to provide a deodorizing composition that effectively reduces body odor, in particular in the axillary region and/or in the region of the feet, and uses well-tolerated, side-effect-free active agents.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

With regard to the masking of body odor, a particular combination of cyclic monoterpene epoxies with substituted aromatic ethers and/or with menthol has proven, surprisingly, to be particularly effective.

A cosmetic composition for use as a deodorant, including components a) to d); a) at least one alkoxybenzene compound having the structural formula (I)

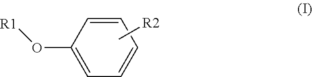

having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %; b) at least one compound selected from cyclic monoterpene epoxies in a total quantity from 0.01 to 1 wt % and menthol in a total quantity from 0.09 to 5 wt %, and from mixtures of said components; c) 0 to 7 wt % water; d) a cosmetically acceptable carrier including at least one component selected from ethanol, a cosmetic oil liquid under standard conditions, and talc, as well as mixtures thereof, as well as optionally further carrier substances, adjuvants, and active agents; wherein the “wt %” indications refer to the total weight of the composition without taking into account any propellant that may be present.

Use of a cosmetic composition including components a) to c); a) at least one alkoxybenzene compound having the structural formula (I)

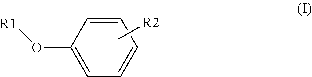

having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %; b) at least one compound selected from cyclic monoterpene epoxies in a total quantity from 0.01 to 1 wt %, and menthol in a total quantity from 0.09 to 5 wt %, and from mixtures of said components; c) a cosmetically acceptable carrier including at least one component selected from water, ethanol, a cosmetic oil liquid under standard conditions, and talc, as well as mixtures thereof, as well as optionally further carrier substances, adjuvants, and active agents; to reduce body odor of the armpits and/or feet, wherein the “wt %” indications refer to the total weight of the composition without taking into account any propellant that may be present.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the present application is cosmetic compositions for use as a deodorant, including components a) to d),

a) at least one alkoxybenzene compound having the structural formula (I)

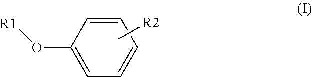

having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %,

b) at least one compound selected from cyclic monoterpene epoxies in a total quantity from 0.01 to 1 wt % and menthol in a total quantity from 0.09 to 5 wt %, and from mixtures of said components,

c) 0 to 7 wt % water,

d) a cosmetically acceptable carrier including at least one component selected from ethanol, a cosmetic oil liquid under standard conditions, and talc, as well as mixtures thereof, as well as optionally further carrier substances, adjuvants, and active agents,

wherein the “wt %” indications refer to the total weight of the composition without taking into account any propellant that may be present.

In preferred compositions according to the present invention and used according to the present invention, the substituent R¹ of the at least one alkoxybenzene compound of the structural formula (I) is selected from a C₁ to C₈ alkyl group, methyl group, ethyl group, n-propyl group, 2-methylethyl group, n-butyl group, n-hexyl group, 2-ethylhexyl group, and an n-octyl group. Compositions according to the present invention and used according to the present invention in which the at least one alkoxybenzene compound having the structural formula (I) is selected from compounds in which R¹ represents a methyl group or an ethyl group, particularly preferably a methyl group, are particularly preferred.

In further preferred compositions according to the present invention and used according to the present invention, the substituent R² of the at least one alkoxybenzene compound of the structural formula (I) is selected from an ethyl group, an n-propyl group, a 1-methylethyl group, an n-butyl group, a 1-propenyl group, and a 2-propenyl group. Compositions according to the present invention and used according to the present invention in which the at least one alkoxybenzene compound having the structural formula (I) is selected from compounds in which R² represents a 1-propenyl group are particularly preferred. Further compositions particularly preferred and preferably used according to the present invention are characterized in that the at least one compound having the structural formula (I) is selected from compounds in which R¹ is a methyl group and R² is a 1-propenyl group.

The substituents OR¹ and R² of the alkoxybenzene compound of the structural formula (I) can be located in the ortho-, meta-, and para- position with respect to one another. Particularly preferred compositions according to the present invention and used according to the present invention are characterized in that the at least one alkoxybenzene compound having the structural formula (I) is selected from those compounds in which the substituents OR¹ and R² are in the para-position with respect to one another.

Further compositions particularly preferred according to the present invention and used according to the present invention are characterized in that the at least one compound having the structural formula (I) is selected from compounds in which R¹ is a methyl group and R² is a 1-propenyl group, wherein the substituents OR¹ and R² are in the para-position with respect to one another. Particularly preferably, these compounds are selected from trans-anethole and from mixtures of cis-anethole and trans-anethole that, based on their weight, include a maximum of 1 wt %, preferably a maximum of 0.5 wt % cis-anethole.

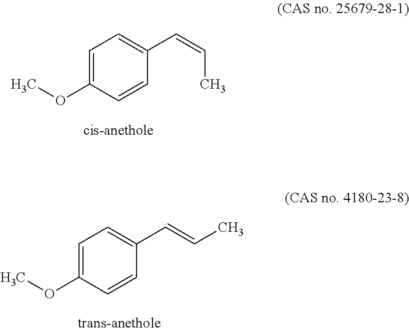

The compositions according to the present invention and used according to the present invention include at least one alkoxybenzene compound of the structural formula (I) in a total quantity from 0.01 to 1 wt %, preferably 0.04 to 0.6 wt %, particularly preferably 0.1 to 0.4 wt %, wherein the “wt %” indications refer to the total weight of the composition without taking into account any propellant that may be present. Compositions preferred and preferably used according to the present invention include 0.01 to 1 wt %, preferably 0.04 to 0.6 wt %, particularly preferably 0.1 to 0.4 wt % trans-anethole, wherein the “wt %” indications refer to the total weight of the composition without taking into account any propellant that may be present.

The compositions according to the present invention and used according to the present invention further include, besides the at least one alkoxybenzene compound of the structural formula (I), at least one compound that is selected from cyclic monoterpene epoxies in a total quantity from 0.01 to 1 wt % and menthol in a total quantity from 0.09 to 5 wt %, and from mixtures of said components, i.e. mixtures of 0.01 to 1 wt % cyclic monoterpene epoxy/ies and 0.09 to 5 wt % menthol, wherein the “wt %” indications refer to the total weight of the composition without taking into account any propellant that may be present.

Compositions preferred and preferably used according to the present invention are characterized in that the at least one cyclic monoterpene epoxy is selected from eucalyptol (=1,8-cineol, 1,8-epoxy-p-menthane, 1,3,3 -trimethyl-2-oxabicyclo[2.2.2]octane) and 1,4-cineol (=1-methyl-4-(1-methylethyl)-7-oxabicyclo[2.2.1]heptane, 1,4-epoxy-p-menthane), and mixtures thereof. A further preferred cyclic monoterpene epoxy is trans-carvone-1,2-epoxy, which is obtainable from the Catasetum species of orchid.

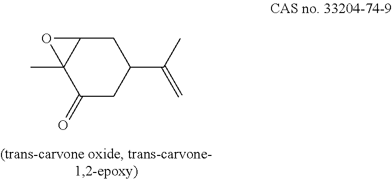

Further compositions preferred and preferably used according to the present invention are characterized in that that at least one cyclic monoterpene epoxy is included in a total quantity from 0.02 to 0.5 wt %, preferably 0.05 to 0.2 wt %, particularly preferably 0.1 to 0.15 wt %, wherein the “wt %” indications refer to the total weight of the composition without taking into account any propellant that may be present. Further compositions preferred and preferably used according to the present invention include 0.01 to 1 wt %, preferably 0.02 to 0.5 wt %, particularly preferably 0.05 to 0.2 wt %, extraordinarily preferably 0.1 to 0.15 wt % eucalyptol, wherein the “wt %” indications refer to the total weight of the composition without taking into account any propellant that may be present. Further compositions preferred and preferably used according to the present invention include 0.01 to 1 wt %, preferably 0.02 to 0.5 wt %, particularly preferably 0.05 to 0.2 wt %, extraordinarily preferably 0.1 to 0.15 wt % 1,4-cineol, wherein the “wt %” indications refer to the total weight of the composition without taking into account any propellant that may be present. Further compositions preferred and preferably used according to the present invention include 0.01 to 1 wt %, preferably 0.02 to 0.5 wt %, particularly preferably 0.05 to 0.2 wt %, extraordinarily preferably 0.1 to 0.15 wt % of a mixture of eucalyptol and 1,4-cineol, wherein the “wt %” indications refer to the total weight of the composition without taking into account any propellant that may be present.

Further compositions preferred and preferably used according to the present invention include 0.01 to 1 wt %, preferably 0.04 to 0.6 wt %, particularly preferably 0.1 to 0.4 wt % trans-anethole and 0.01 to 1 wt %, preferably 0.02 to 0.5 wt %, particularly preferably 0.05 to 0.2 wt %, extraordinarily preferably 0.1 to 0.15 wt % eucalyptol, wherein the “wt %” indications refer to the total weight of the composition without taking into account any propellant that may be present.

Compositions preferred and preferably used according to the present invention are characterized in that the menthol is selected from L-menthol, D-menthol, and DL-menthol, preferably selected from DL-menthol. Further compositions preferred and preferably used according to the present invention include 0.09 to 5 wt %, preferably 0.1 to 2.5 wt %, particularly preferably 0.25 to 1.8 wt %, extraordinarily preferably 0.5 to 1.0 wt % menthol, selected from L-menthol, D-menthol, and DL-menthol, wherein the “wt %” indications refer to the total weight of the composition without taking into account any propellant that may be present. Compositions particularly preferred according to the present invention include 0.01 to 1 wt %, preferably 0.04 to 0.6 wt %, particularly preferably 0.1 to 0.4 wt % trans-anethole and 0.09 to 5 wt %, preferably 0.1 to 2.5 wt %, particularly preferably 0.25 to 1.8 wt %, extraordinarily preferably 0.5 to 1.0 wt % menthol, wherein the “wt %” indications refer to the total weight of the composition without taking into account any propellant that may be present. Further compositions particularly preferred according to the present invention include 0.01 to 1 wt %, preferably 0.04 to 0.6 wt %, particularly preferably 0.1 to 0.4 wt % trans-anethole and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present. Further compositions particularly preferred and particularly preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % menthol, wherein the “wt %” indications refer to the total weight of the composition without taking into account any propellant that may be present. Further compositions particularly preferred and particularly preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present.

Further compositions preferred and preferably used according to the present invention are characterized in that the weight ratio of all alkoxybenzene compounds having the structural formula (I) according to claim 1 to menthol is in the range from 1:0.8 to 1:10, preferably 1:1 to 1:7, particularly preferably 1:2 to 1:4.

Further compositions preferred and preferably used according to the present invention are characterized in that the weight ratio of all alkoxybenzene compounds having the structural formula (I) according to claim 2 to menthol is in the range from 1:0.8 to 1:10, preferably 1:1 to 1:7, particularly preferably 1:2 to 1:4.

Further compositions preferred and preferably used according to the present invention are characterized in that the weight ratio of all alkoxybenzene compounds having the structural formula (I) according to claim 3 to menthol is in the range from 1:0.8 to 1:10, preferably 1:1 to 1:7, particularly preferably 1:2 to 1:4.

Further compositions preferred and preferably used according to the present invention are characterized in that the weight ratio of all alkoxybenzene compounds having the structural formula (I) according to claim 4 to menthol is in the range from 1:0.8 to 1:10, preferably 1:1 to 1:7, particularly preferably 1:2 to 1:4.

Further compositions preferred and preferably used according to the present invention are characterized in that the weight ratio of all alkoxybenzene compounds having the structural formula (I) according to claim 1 to cyclic monoterpene epoxies is in the range from 1:0.2 to 1:1.1, preferably 1:0.5 to 1:1, particularly preferably 1:0.6 to 1:0.8.

Further compositions preferred and preferably used according to the present invention are characterized in that the weight ratio of all alkoxybenzene compounds having the structural formula (I) according to claim 2 to cyclic monoterpene epoxies is in the range from 1:0.2 to 1:1.1, preferably 1:0.5 to 1:1, particularly preferably 1:0.6 to 1:0.8.

Further compositions preferred and preferably used according to the present invention are characterized in that the weight ratio of all alkoxybenzene compounds having the structural formula (I) according to claim 3 to cyclic monoterpene epoxies is in the range from 1:0.2 to 1:1.1, preferably 1:0.5 to 1:1, particularly preferably 1:0.6 to 1:0.8.

Further compositions preferred and preferably used according to the present invention are characterized in that the weight ratio of all alkoxybenzene compounds having the structural formula (I) according to claim 4 to cyclic monoterpene epoxies is in the range from 1:0.2 to 1:1.1, preferably 1:0.5 to 1:1, particularly preferably 1:0.6 to 1:0.8.

Further compositions preferred and preferably used according to the present invention are characterized in that the weight ratio of all alkoxybenzene compounds having the structural formula (I) according to claim 1 to the sum of cyclic monoterpene epoxies and menthol is in the range from 1:1 to 1:10, preferably 1:2 to 1:7, particularly preferably 1:4 to 1:6.

Further compositions preferred and preferably used according to the present invention are characterized in that the weight ratio of all alkoxybenzene compounds having the structural formula (I) according to claim 2 to the sum of cyclic monoterpene epoxies and menthol is in the range from 1:1 to 1:10, preferably 1:2 to 1:7, particularly preferably 1:4 to 1:6.

Further compositions preferred and preferably used according to the present invention are characterized in that the weight ratio of all alkoxybenzene compounds having the structural formula (I) according to claim 3 to the sum of cyclic monoterpene epoxies and menthol is in the range from 1:1 to 1:10, preferably 1:2 to 1:7, particularly preferably 1:4 to 1:6.

Further compositions preferred and preferably used according to the present invention are characterized in that the weight ratio of all alkoxybenzene compounds having the structural formula (I) according to claim 4 to the sum of cyclic monoterpene epoxies and menthol is in the range from 1:1 to 1:10, preferably 1:2 to 1:7, particularly preferably 1:4 to 1:6.

Further compositions particularly preferred and preferably used according to the present invention include 0.01 to 1 wt %, preferably 0.04 to 0.6 wt %, particularly preferably 0.1 to 0.4 wt % trans-anethole and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of trans-anethole to menthol is in the range from 1:0.8 to 1:10, preferably 1:1 to 1:7, particularly preferably 1:2 to 1:4.

Further compositions particularly preferred and preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of trans-anethole to menthol is in the range from 1:0.8 to 1:10, preferably 1:1 to 1:7, particularly preferably 1:2 to 1:4.

Further compositions particularly preferred and preferably used according to the present invention include 0.01 to 1 wt %, preferably 0.04 to 0.6 wt %, particularly preferably 0.1 to 0.4 wt % trans-anethole and 0.01 to 1 wt %, preferably 0.02 to 0.5 wt %, particularly preferably 0.05 to 0.2 wt %, extraordinarily preferably 0.1 to 0.15 wt % eucalyptol, wherein the “wt %” indications refer to the total weight of the composition without taking into account any propellant that may be present, wherein the weight ratio of trans-anethole to eucalyptol is in the range from 1:0.2 to 1:1.1, preferably 1:0.5 to 1:1, particularly preferably 1:0.6 to 1:0.8.

Further compositions particularly preferred and preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of trans-anethole to eucalyptol is in the range from 1:0.2 to 1:1.1, preferably 1:05 to 1:1, particularly preferably 1:0.6 to 1:0.8.

Further compositions particularly preferred and preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of menthol to eucalyptol is in the range from 1:1 to 10:1, preferably 2:1 to 8:1, particularly preferably 3:1 to 5:1.

Further compositions particularly preferred and preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of menthol to eucalyptol is in the range from 1:1 to 20:1, preferably 5:1 to 15:1, particularly preferably 7:1 to 10:1.

Further compositions particularly preferred and preferably used according to the present invention include 0.01 to 1 wt %, preferably 0.04 to 0.6 wt %, particularly preferably 0.1 to 0.4 wt % trans-anethole, 0.01 to 1 wt %, preferably 0.02 to 0.5 wt %, particularly preferably 0.05 to 0.2 wt %, extraordinarily preferably 0.1 to 0.15 wt % eucalyptol, and 0.09 to 5 wt %, preferably 0.1 to 2.5 wt %, particularly preferably 0.25 to 1.8 wt %, extraordinarily preferably 0.5 to 1.0 wt % DL-menthol, wherein the “wt %” indications refer to the total weight of the composition without taking into account any propellant that may be present, wherein the weight ratio of trans-anethole to the sum of eucalyptol and menthol is in the range from 1:1 to 1:10, preferably 1:2 to 1:7, particularly preferably 1:4 to 1:6.

The compositions according to the present invention include 0 to 7 wt %, preferably 0 to 5 wt %, particularly preferably 0 to 3 wt %, extraordinarily preferably 0 to 1 wt % water, based in each case on the weight of the composition without taking into account any propellant that may be included.

Compositions used according to the present invention to reduce body odor of the armpits and/or the feet include 0 to 90 wt %, preferably 3 to 80 wt %, particularly preferably 5 to 75 wt %, extraordinarily preferably 10 to 70 wt % water, more preferably 30 to 60 wt % water, and in particular 40 to 55 wt % water, based in each case on the weight of the composition used according to the present invention without taking into account any propellant that may be included.

In a further preferred embodiment, the compositions used according to the present invention include 10 to 90 wt %, preferably 25 to 80 wt %, more preferably 30 to 75 wt %, more preferably 40 to 70 wt %, and in particular 50 to 65 wt % water, as well as at least one water-soluble polyol from the group of polyols having 2 to 9 carbon atoms and 2 to 6 hydroxyl groups, in a total quantity from 1 to 40 wt %, preferably 2 to 25 wt %, more preferably 4 to 15 wt %, and in particular 5 to 10 wt %, based in each case on the weight of the composition used according to the present invention without taking into account any propellant that may be included.

Preferred water-soluble polyols from the group of polyols having 2 to 9 carbon atoms and 2 to 6 hydroxyl groups, which are not 1,2-hexanediol and not 1,2-octanediol, are selected from 1,2-propanediol, diethylene glycol, 2-methyl-1,3-propanediol, glycerol, 1,2-butylene glycol, 1,3-butylene glycol, 1,4-butylene glycol, 1,2-pentanediol, 1,5-pentanediol, 1,6-hexanediol, 1,2,6-hexanetriol, dipropylene glycol, tripropylene glycol, diglycerol, triglycerol, polyglycerol, erythritol, sorbitol, methylglucoside, butylglucoside, trans-1,4-dimethylolcyclohexane, cis-1,4-dimethylolcyclohexane, and mixtures of the aforementioned substances.

In a further preferred embodiment, the compositions used according to the present invention include 10 to 90 wt %, preferably 25 to 80 wt %, more preferably 30 to 75 wt %, more preferably 40 to 70 wt %, and in particular 50 to 65 wt % water, as well as ethanol in a quantity from 1 to 90 wt %, preferably 5 to 85 wt %, particularly preferably 10 to 75 wt %, extraordinarily preferably 20 to 50 wt %, based in each case on the weight of the composition used according to the present invention without taking into account any propellant that may be included.

The compositions according to the present invention include a cosmetically acceptable carrier that comprises at least one component selected from ethanol, a cosmetic oil liquid under standard conditions, and talc, as well as mixtures thereof.

The compositions used according to the present invention include a cosmetically acceptable carrier that comprises at least one component selected from water, ethanol, a cosmetic oil liquid under standard conditions, and talc, as well as mixtures thereof.

In a preferred embodiment, the compositions according to the present invention include 0 to 5 wt %, preferably 0 to 3 wt %, particularly preferably 0 to 1 wt % water, as well as ethanol in a quantity from 1 to 90 wt %, preferably 5 to 85 wt %, particularly preferably 10 to 75 wt %, extraordinarily preferably 25 to 60 wt %, based in each case on the weight of the composition without taking into account any propellant that may be included.

“Standard conditions” for purposes of the present application are a temperature of 20° C. and a pressure of 1013.25 mbar. Melting-point indications likewise refer to a pressure of 1013.25 mbar.

The total quantity of cosmetic oils liquid under standard conditions in compositions preferred and preferably used according to the present invention is 1 to 95 wt %, preferably 5 to 90 wt %, particularly preferably 30 to 75 wt %, extraordinarily preferably 50 to 60 wt %, wherein the quantity indications refer to the weight of the composition without taking in to account any propellant that may be present.

A distinction is made in the context of cosmetic oils between volatile and nonvolatile oils. “Nonvolatile” oils are understood as those oils that, at 20° C. and an ambient pressure of 1013 hPa, have a vapor pressure of less than 2.66 Pa (0.02 mm Hg). “Volatile” oils are understood as those oils that, at 20° C. and an ambient pressure of 1013 hPa, have a vapor pressure from 2.66 Pa to 40,000 Pa (0.02 mm to 300 mm Hg), preferably 13 to 12,000 Pa (0.1 to 90 mm Hg), particularly preferably 15 to 8000 Pa, extraordinarily preferably 200 to 3000 Pa.

Volatile cosmetic oils are usually selected from among cyclic silicone oils having the INCI name Cyclomethicones. The INCI name Cyclomethicone is understood in particular to mean cyclotrisiloxane (hexamethylcyclotrisiloxane), cyclotetrasiloxane (octamethylcyclotetrasiloxane), cyclopentasiloxane (decamethylcyclopentasiloxane), and cyclohexasiloxane (dodecamethylcyclohexasiloxane). These oils have a vapor pressure of approx. 13 to 15 Pa at 20° C.

Cyclomethicones are known in the existing art as oils well suited for cosmetic compositions, in particular for deodorizing compositions such as sprays and sticks. Because of their persistence in the environment, however, it can be preferred according to the present invention to omit the use of cyclomethicones. In an especially preferred embodiment, the compositions according to the present invention and used according to the present invention include 0 to less than 1 wt % cyclomethicone, based on the weight of the composition, wherein any propellant that may be present is not taken into account.

Further preferred volatile silicone oils are selected from volatile linear silicone oils, in particular volatile linear silicone oils having 2 to 10 siloxane units, such as hexamethyldisiloxane (L2), octamethyltrisiloxane (L₃), decamethyltetrasiloxane (L₄), as included e.g. in the commercial products DC 2-1184, Dow Corning® 200 (0.65 cSt), and Dow Corning® 200 (1.5 cSt) of Dow Corning, and low-molecular-weight Phenyl Trimethicone having a vapor pressure at 20° C. of approximately 2000 Pa, as obtainable e.g. from GE Bayer Silicones/Momentive under the name Baysilone Fluid PD 5.

Further compositions preferred and preferably used according to the present invention include, because of the drier skin feel, at least one volatile non-silicone oil. Preferred volatile non-silicone oils are selected from C₈ to C₁₆ isoparaffins, in particular from isononane, isodecane, isoundecane, isododecane, isotridecane, isotetradecane, isopentadecane, and isohexadecane, as well as mixtures thereof. C₁₀ to C₁₃ isoparaffin mixtures, in particular those having a vapor pressure at 20° C. from approximately 300 to 400 Pa, preferably 360 Pa, are preferred. This at least one C₈ to C₁₆ isoparaffin is included preferably in a total quantity from 25 to 50 wt %, preferably 30 to 45 wt %, particularly preferably 32 to 40 wt %, extraordinarily preferably 34 to 37 wt %, based in each case on the total propellant-free composition.

In compositions preferred and preferably used according to the present invention the at least one oil liquid under standard conditions comprises at least one volatile C₈ to C₁₆ isoparaffin, in particular isononane, isodecane, isoundecane, isododecane, isotridecane, isotetradecane, isopentadecane, and isohexadecane, as well as mixtures thereof.

Further compositions preferred and preferably used according to the present invention include at least one nonvolatile cosmetic oil selected from nonvolatile silicone oils and nonvolatile non-silicone oils. Residua of constituents insoluble in the carrier, such as antiperspirant active agents or talc, can be successfully masked with a nonvolatile oil. In addition, using a mixture of various oils, in particular of nonvolatile and volatile oil, parameters such as skin feel, visibility of residua, and stability of the composition according to the present invention can be precisely regulated and better adapted to consumers' needs.

It is of course likewise possible to formulate compositions according to the present invention and used according to the present invention having a small proportion of volatile oils—i.e. having 0.5 to 24.5 wt % volatile oils, based on the weight of the propellant-free agent—or even having no volatile oils.

Oils particularly preferred according to the present invention are esters of linear or branched, saturated or unsaturated fatty alcohols having 2 to 30 carbon atoms with linear or branched, saturated or unsaturated fatty acids having 2 to 30 carbon atoms, which can be hydroxylated. Be it noted in this regard that some esters of linear or branched C₁ to C₂₂ alkanols or C₁₄ to C₂₂ alkenols, and some triesters of glycerol with linear or branched C₂ to C₂₂ carboxylic acids, which can be saturated or unsaturated, are solid under standard conditions, such as e.g. cetyl stearate or glycerol tristearate (=stearin). These esters that are solid under standard conditions do not represent cosmetic oils according to the present invention, since of course they do not meet the criterion of “liquid under standard conditions.” The categorization as to whether such an ester is liquid or solid under standard conditions is a matter of the skilled artisan's general knowledge.

Esters of linear or branched saturated fatty alcohols having 2 to 5 carbon atoms with linear or branched, saturated or unsaturated fatty acids having 3 to 18 carbon atoms, which can be hydroxylated, are preferred. Preferred examples thereof are isopropyl palmitate, isopropyl stearate, isopropyl myristate, 2-hexyldecyl stearate, 2-hexyldecyl laurate, isononyl isononanoate, 2-ethylhexyl palmitate, and 2-ethylhexyl stearate. Also preferred are isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid 2-butyl octanoate, diisotridecyl acetate, n-hexyl laurate, n-decyl oleate, oleyl oleate, oleyl erucate, erucyl oleate, triethyl citrate, C₁₂ to C₁₅ alkyl lactate, and di-C₁₂ to C₁₃ alkyl malate, as well as benzoic acid esters of linear or branched C₈₋₂₂ alkanols. Benzoic acid C₁₂ to C₁₅ alkyl esters are particularly preferred, obtainable e.g. as a commercial product Finsolv® TN (C₁₂ to C₁₅ alkyl benzoate), as well as benzoic acid isostearyl esters, obtainable e.g. as Finsolv® SB, 2-ethylhexyl benzoate, obtainable e.g. as Finsolv® EB, and benzoic acid 2-octyldodecyl esters, obtainable e.g. as Finsolv® BOD.

Further nonvolatile non-silicone oils preferred according to the present invention are selected from branched saturated or unsaturated fatty alcohols having 6 to 30 carbon atoms. These alcohols are often referred to as “Guerbet alcohols,” since they are obtainable according to the Guerbet reaction. Preferred alcohol oils are 2-hexyldecanol, 2-octyldodecanol, and 2-ethylhexyl alcohol. Isostearyl alcohol is likewise preferred. Further preferred nonvolatile oils are selected from mixtures of Guerbet alcohols and Guerbet alcohol esters, for example 2-hexyldecanol and 2-hexyldecyl laurate.

The expression “triglyceride” used below means “glycerol triester.” Further nonvolatile oils preferred according to the present invention are selected from triglycerides of linear or branched, saturated or unsaturated, optionally hydroxylated C₈₋₃₀ fatty acids, provided they are liquid under standard conditions. The use of natural oils, e.g. soy oil, cottonseed oil, sunflower oil, palm oil, palm kernel oil, linseed oil, almond oil, castor oil, corn oil, rapeseed oil, olive oil, sesame oil, thistle oil, wheat germ oil, peach-kernel oil, and the liquid components of coconut oil and the like, can be particularly suitable. Synthetic triglyceride oils are particularly preferred, in particular Capric/Caprylic Triglycerides, e.g. the commercial products Myritol® 318 or Myritol® 331 (BASF/Cognis) having unbranched fatty acid esters, as well as glyceryl triisostearin and glyceryl tri(2-ethylhexanoate) having branched fatty acid esters. Triglyceride oils of this kind preferably account for a proportion of less than 50 wt % of the total weight of all cosmetic oils in the composition according to the present invention. Particularly preferably, the total weight of triglyceride oils is 0.5 to 10 wt %, preferably 1 to 5 wt %, based in each case on the total composition without taking into account any propellant that may be present.

Further nonvolatile non-silicone oils particularly preferred according to the present invention are selected from dicarboxylic acid esters of linear or branched C₂ to C₁₀ alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate.

Further nonvolatile non-silicone oils particularly preferred according to the present invention are selected from addition products of 1 to 5 propylene oxide units with mono- or polyvalent C₈₋₂₂ alkanols such as octanol, decanol, decanediol, lauryl alcohol, myristyl alcohol, and stearyl alcohol, e.g. PPG-2 Myristyl Ether and PPG-3 Myristyl Ether.

Further nonvolatile non-silicone oils particularly preferred according to the present invention are selected from the addition products of at least 6 ethylene oxide and/or propylene oxide units with mono- or polyvalent C₃₋₂₂ alkanols such as glycerol, butanol, butanediol, myristyl alcohol, and stearyl alcohol, which can be esterified if desired, e.g. PPG-14 Butyl Ether, PPG-9 Butyl Ether, PPG-10 Butanediol, PPG-15 Stearyl Ether, and Glycereth-7 Diisononanoate.

Further nonvolatile non-silicone oils particularly preferred according to the present invention are selected from symmetrical, asymmetrical, or cyclic esters of carbonic acid with C₆ to C₂₀ alcohols, e.g. di-n-caprylyl carbonate (Cetiol® CC), or di-(2-ethylhexyl) carbonate (Tegosoft DEC). Esters of carbonic acid with C₁ to C₅ alcohols, however, e.g. glycerol carbonate or propylene carbonate, are not compounds suitable as a cosmetic oil.

Further oils that can be preferred according to the present invention are selected from esters of dimers of unsaturated C₁₂ to C₂₂ fatty acids (dimer fatty acids) with monovalent linear, branched, or cyclic C₂ to C₁₈ alkanols or with polyvalent linear or branched C₂ to C₆ alkanols. The total weight of dimer fatty acid esters is particularly preferably 0.5 to 10 wt %, preferably 1 to 5 wt %, based in each case on the total composition without taking into account any propellant that may be present.

Compositions according to the present invention and used according to the present invention can also be formulated as deodorizing body powders. The principal constituent of such carriers is talc. Body powders according to the present invention and used according to the present invention can be present as a loose powder or a compact powder. The powder base for loose powders usually comprises at least 70 wt % talc, 2 to 10 wt % metal soaps, such as in particular the stearates of magnesium, zinc, titanium, calcium, and aluminum, preferably magnesium stearate, in addition further powdered constituents selected from silicon dioxide, starch, titanium dioxide, zinc dioxide, kaolin, calcium carbonate, and magnesium carbonate. Compact powders include talc usually in quantities of less than 70 wt %, for example 5 to 60 wt %, and additionally oils and/or waxes in a quantity from 2 to 15 wt %.

The compositions according to the present invention and used according to the present invention optionally include further carrier substances, adjuvants, and active agents.

In a further preferred embodiment the agents according to the present invention and used according to the present invention include as a deodorizing active substance, in addition to the active-agent combination according to the present invention, at least one aromatic alcohol of structure (AA-1)

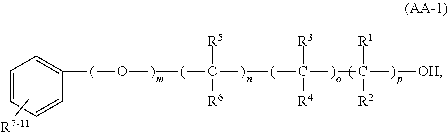

wherein
residues R¹ to R⁶ mutually independently denote a hydrogen atom, an alkyl group having 1 to 10 carbon atoms, which can be linear or branched and can be substituted with OH groups or alkoxy groups having 1 to 5 carbon atoms, or an alkenyl group having 2 to 10 carbon atoms, which can be linear or branched and can be substituted with OH groups or alkoxy groups having 1 to 5 carbon atoms,
residues R⁷ to R¹¹ mutually independently denote a hydrogen atom, a halogen atom, in particular a chlorine atom, or an alkyl group having 1 to 10 carbon atoms, which can be linear or branched and can be substituted with OH groups or alkoxy groups having 1 to 5 carbon atoms, in particular with a methoxy group,
m=0 or 1, n, o, p mutually independently are integers from 0 to 10, at least one of the values n, o, p being not equal to 0.

Particularly preferred compositions according to the present invention and used according to the present invention include at least one alcohol AA-1 as described above which is selected from anise alcohol, 2-methyl-5-phenylpentan-1-ol, 1,1-dimethyl-3-phenylpropan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2,2-dimethyl-3-phenylpropan-1-ol, 2,2-dimethyl-3-(3′-methylphenyl)propan-1-ol, 2-ethyl-3-phenylpropan-1-ol, 2-ethyl-3-(3′-methylphenyl)propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-ol, 2-ethyl-3-(2′-methylphenyl)propan-1-ol, 2-ethyl-3-(4′-methylphenyl)propan-1-ol, 3-(3′,4′-dimethylphenyl)-2-ethylpropan-1-ol, 2-ethyl-3-(4′-methoxyphenyl)propan-1-ol, 3-(3′,4′-dimethoxyphenyl)-2-ethylpropan-1-ol, 2-allyl-3-phenylpropan-1-ol, and 2-n-pentyl-3-phenylpropan-1-ol, as well as mixtures thereof. 2-Benzylheptan- 1-ol, as well as mixtures of 2-benzylheptan- 1-ol and phenoxyethanol, are extraordinarily preferred.

Further particularly preferred compositions according to the present invention and used according to the present invention include at least one alcohol AA-1 as described above in a total quantity from 0.05 to 10 wt %, preferably 0.1 to 5 wt %, particularly preferably 0.2 to 2 wt %, extraordinarily preferably 0.3 to 1.5 wt %, based in each case on the weight of the composition without taking into account any propellant that may be included.

Further particularly preferred compositions according to the present invention and used according to the present invention are characterized by including the deodorizing active agent 3-(2-ethylhexyloxy)-1,2-propanediol, preferably in a total quantity from 0.05 to 5 wt %, preferably 0.1 to 2 wt %, particularly preferably 0.2 to 1.5 wt %, extraordinarily preferably 0.5 to 1.0 wt %, based in each case on the weight of the composition without taking into account any propellant that may be included.

Further particularly preferred compositions according to the present invention and used according to the present invention are characterized by including tropolone (2-hydroxy-2,4,6-cycloheptatrienone), preferably in a quantity from 0.001 to 0.1 wt %, based on the weight of the composition without taking into account any propellant that may be included.

Further particularly preferred compositions according to the present invention and used according to the present invention are characterized by including 1,2-hexanediol and/or 1,2-octanediol as highly effective deodorant active agents that do not disrupt the microbial balance of healthy skin. Preferred deodorant compositions according to the present invention and used according to the present invention include 0.1 to 1.0 wt % 1,2-hexanediol and/or 0.1 to 10 wt % 1,2-octanediol, based in each case on the weight of the composition without taking into account any propellant that may be included. Particularly preferred deodorant compositions according to the present invention include 0.1 to 5 wt %, preferably 0.2 to 1 wt % 1,2-hexanediol and/or 0.1 to 5 wt %, preferably 0.2 to 1 wt % 1,2-octanediol, based in each case on the weight of the composition without taking into account any propellant that may be included. Extraordinarily preferred deodorant compositions according to the present invention and used according to the present invention include 0.2 to 0.5 wt % 1,2-hexanediol and 0.2 to 0.5 wt % 1,2-octanediol, based in each case on the weight of the composition without taking into account any propellant that may be included.

Further particularly preferred compositions according to the present invention and used according to the present invention are characterized by including triethyl citrate. Triethyl citrate is a known deodorant active agent that acts as an enzyme inhibitor for esterases and lipases and thus contributes to the broadband action of compositions preferred according to the present invention. Preferred compositions according to the present invention and used according to the present invention include 0.5 to 15 wt %, preferably 3 to 8 wt %, extraordinarily preferably 4 to 6 wt %, based in each case on the weight of the composition without taking into account any propellant that may be included.

Further particularly preferred compositions according to the present invention and used according to the present invention are characterized by including at least one cationic phospholipid of formula KPL

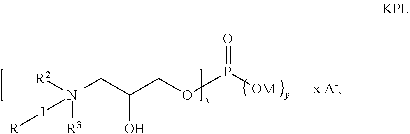

in which R¹ is an alkyl, alkenyl, or hydroxyalkyl group having 8 to 22 carbon atoms or an acylaminoalkyl group of the formula R⁵CONH(CₘH₂ₘ)— in which R⁵CO is a linear acyl group having 8 to 22 carbon atoms and m=2 or 3,
R² and R³ are alkyl groups having 1 to 4 carbon atoms or hydroxyalkyl groups having 2 to 4 carbon atoms or carboxyalkyl groups of the formula —(CH₂)_{z}—COOM, in which z has a value from 1 to 3 and M is hydrogen or an alkali metal cation,
x has a value from 1 to 3 and y a value of (3-x), M is hydrogen or an alkali metal cation, and A⁻ is an anion.

Preferred alkyl groups having 8 to 22 carbon atoms are selected from an n-octyl, n-nonyl, n-decyl, n-undecyl, lauryl, n-tridecanyl, myristyl, n-pentadecanyl, cetyl, palmityl, stearyl, elaidyl, arachidyl, behenyl, and a cocyl group. A representative cocyl group is made up, based on its total weight, of 4 to 9 wt % n-octyl groups, 4 to 9 wt % n-decyl groups, 45 to 55 wt % lauryl groups, 15 to 21 wt % myristyl groups, 8 to 13 wt % palmityl groups, and 7 to 14 wt % stearyl groups. Preferred alkenyl groups having 8 to 22 carbon atoms are selected from a linoleyl group ((9Z,12Z)-octadeca-9,12-dien-1-yl) and a linolenyl group ((9Z,12Z,15Z)-octadeca-9,12,15-trien-1-yl). A preferred hydroxyalkyl group having 8 to 22 carbon atoms is selected from a 12-hydroxystearyl group.

Particularly preferred cationic phospholipids of formula KPL are those in which R¹ is an acylaminoalkyl group of the formula R⁵CONH(CₘH₂ₘ)−, in which R⁵CO represents a linear acyl group having 8 to 22 carbon atoms and m=3.

Preferred linear acyl groups R⁵CO having 8 to 22 carbon atoms are selected from an n-octanoyl, n-nonanoyl, n-decanoyl, n-undecanoyl, lauroyl, n-tridecanoyl, myristoyl, n-pentadecanoyl, cetoyl, palmitoyl, stearoyl, elaidoyl, arachidoyl, behenoyl, and a cocoyl group. A representative cocoyl group is made up, based on its total weight, of 4 to 9 wt % n-octanoyl groups, 4 to 9 wt % n-decanoyl groups, 45 to 55 wt % lauroyl groups, 15-21 wt % myristoyl groups, 8 to 13 wt % palmitoyl groups, and 7 to 14 wt % stearoyl groups. Particularly preferred linear acyl groups R⁵CO are selected from a cocoyl group, a lauroyl group (n-C₁₁H₂₃CO), a myristoyl group (n-C₁₃H₂₇CO), and a linoleoyl group ((9Z,12Z)-octadeca-9,12-dien-1-oyl). Extraordinarily preferred linear acyl groups R⁵CO are selected from a cocoyl group, a lauroyl group (n-C₁₁H₂₃CO), and a myristoyl group (n-C₁₃H₂₇CO).

Preferred alkyl groups having 1 to 4 carbon atoms are a methyl, ethyl, n-propyl, 1-methylethyl, n-butyl, 2-methylpropyl, and tert-butyl group. The methyl group is particularly preferred.

Preferred hydroxyalkyl groups having 2 to 4 carbon atoms are a 2-hydroxyethyl group and a 1-hydroxyethyl group.

Preferred carboxyalkyl groups of the formula —(CH₂)_{z}—COOM, where z=1 to 3, are a carboxymethyl, a carboxyethyl, and a carboxy-n-propyl group.

Preferred alkali metal cations are selected from sodium and potassium cations; Na⁺ is particularly preferred. Preferred anions are selected from sulfate, chloride, phosphate, nitrate, hydrogen carbonate, and acetate, a chloride anion being particularly preferred.

Particularly preferred compositions according to the present invention include as a deodorizing active substance a cationic phospholipid of formula KPL

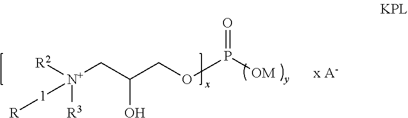

in which R¹ is an acylaminoalkyl group of the formula R⁵CONH(CₘH₂ₘ)—, in which R⁵CO is selected from a cocoyl group, a lauroyl group, a myristoyl group, and a linoleoyl group, and m=3, R² and R³ are methyl groups, x=2, y=1, M is a sodium ion, and A⁻ is a chloride ion.

Preferably at least one cationic phospholipid of formula KPL having the features recited above is included in a total quantity from 0.05 to 2 wt %, preferably 0.1 to 1 wt %, particularly preferably 0.15 to 0.4 wt %, based in each case on the weight of the composition without taking into account any propellant that may be included.

Particularly preferred compositions according to the present invention include a cationic phospholipid of formula KPL

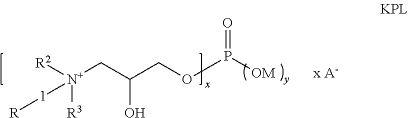

in which R¹ is a cocoylaminopropyl group (also referred to as a cocamidopropyl group), R² and R³ are methyl groups, x=2, y=1, M is a sodium ion, and A³¹ is a chloride ion, and which is obtainable under the INCI name Cocamidopropyl PG-Dimonium Chloride Phosphate, in a total quantity from 0.05 to 2 wt %, preferably 0.1 to 1 wt %, particularly preferably 0.15 to 0.4 wt %, based in each case on the weight of the composition without taking into account any propellant that may be included.

Further particularly preferred compositions according to the present invention and used according to the present invention include a cationic phospholipid of formula KPL

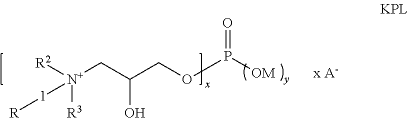

in which R¹ is a myristoylaminopropyl group, R² and R³ are methyl groups, x=2, y=1, M is a sodium ion, and A³¹ is a chloride ion, and which is obtainable under the INCI name Myristoamidopropyl PG-Dimonium Chloride Phosphate, in a total quantity from 0.05 to 2 wt %, preferably 0.1 to 1 wt %, particularly preferably 0.15 to 0.4 wt %, based in each case on the weight of the composition without taking into account any propellant that may be included.

Further particularly preferred compositions according to the present invention include a cationic phospholipid of formula KPL

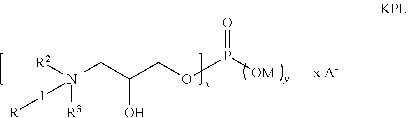

in which R¹ is a lauroylaminopropyl group, R² and R³ are methyl groups, x=2, y=1, M is a sodium ion, and A⁻ is a chloride ion, in a total quantity from 0.05 to 2 wt %, preferably 0.1 to 1 wt %, particularly preferably 0.15 to 0.4 wt %, based in each case on the weight of the composition without taking into account any propellant that may be included.

The compositions according to the present invention and used according to the present invention can also include perspiration-inhibiting active agents, in particular perspiration-inhibiting aluminum salts and aluminum-zirconium salts.

Preferred antiperspirant active agents are selected from aluminum salts, preferably from water-soluble astringent inorganic and organic salts of aluminum and of aluminum-zirconium mixtures. Aluminosilicates and zeolites are not included according to the present invention among the antiperspirant active substances.

“Water solubility” is understood according to the present invention as a solubility of at least 3 wt % at 20° C., i.e. quantities of at least 3 g of the antiperspirant active substance are soluble in 97 g water at 20° C.

Particularly preferred antiperspirant active agents are selected from aluminum chlorohydrate, in particular aluminum chlorohydrate having the general formula [Al₂(OH)₅Cl.1-6H₂O]ₙ, preferably [Al₂(OH)₅Cl.2-3H₂O]ₙ, which can be present in nonactivated or activated (depolymerized) form, as well as aluminum chlorohydrate having the general formula [Al₂(OH)₄Cl₂.1-6H₂O]ₙ, preferably [Al₂(OH)₄Cl₂.2-3H₂O]ₙ, which can be present in nonactivated or activated (depolymerized) form.

The manufacture of preferred antiperspirant active agents is disclosed, for example, in U.S. Pat. No. 3,887,692, U.S. Pat. No. 3,904,741, U.S. Pat. No. 4,359,456, GB 2048229, and GB 1347950.

Also preferred are aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, furthermore selected from aluminum zirconium chlorohydrates, such as aluminum zirconium trichlorohydrate, aluminum zirconium tetrachlorohydrate, aluminum zirconium pentachlorohydrate, aluminum zirconium octachlorohydrate, aluminum zirconium chlorohydrate glycine complexes, such as aluminum zirconium trichlorohydrex glycine, aluminum zirconium tetrachlorohydrex glycine, aluminum zirconium pentachlorohydrex glycine, aluminum zirconium octachlorohydrex glycine, further selected from potassium aluminum sulfate having zero to 12 parts water of crystallization (KAl(SO₄)₂.0H₂O, KAl(SO₄)₂.1H₂O, KAl(SO₄)₂.2H₂O, KAl(SO₄)₂.3H₂O, KAl(SO₄)₂.4H₂O, KAl(SO₄)₂.5 H₂O, KAl(SO₄)₂.6H₂O, KAl(SO₄)₂.7H₂O, KAl(SO₄)₂.8H₂O, KAl(SO₄)₂.9H₂O, KAl(SO₄)₂.10H₂O, KAl(SO₄)₂.11H₂O, KAl(SO₄)₂.12H₂O=alum, partly hydrogenated alum or calcined alum), aluminum undecylenoyl collagen amino acid, sodium aluminum lactate+aluminum sulfate, sodium aluminum chlorohydroxylactate, aluminum bromohydrate, aluminum chloride, aluminum salts of lipoamino acids, aluminum sulfate, aluminum lactate, aluminum chlorohydroxyallantoinate, and sodium aluminum chlorohydroxylactate.

Antiperspirant active agents particularly preferred according to the present invention are selected from so-called “activated” aluminum and aluminum-zirconium salts, which are also referred to as “enhanced activity” antiperspirant active agents. Such active agents are known in the existing art and also commercially obtainable. Their manufacture is disclosed, for example, in GB 2048229, U.S. Pat. No. 4,775,528, and U.S. Pat. No. 6,010,688. Activated aluminum and aluminum-zirconium salts are generally produced by heat treatment of a relatively dilute solution of the salt (e.g. approximately 10 wt % salt), in order to increase its HPLC peak 4 to peak 3 area ratio. The activated salt can then be dried, in particular spray-dried, to a powder. Besides spray drying, drum drying is, for example, also suitable.

Activated aluminum and aluminum-zirconium salts typically have an HPLC peak 4 to peak 3 area ratio of at least 0.4, preferably at least 0.7, particularly preferably at 0.9, where at least 70% of the aluminum is to be associated with these peaks.

Activated aluminum and aluminum-zirconium salts do not necessarily need to be used as a spray-dried powder. Perspiration-inhibiting active substances that are likewise preferred according to the present invention are nonaqueous solutions or solubilizates of an activated perspiration-inhibiting aluminum or aluminum-zirconium salt, for example in accordance with U.S. Pat. No. 6,010,688, which are stabilized against loss of activation (rapid decrease in the HPLC peak 4 to peak 3 area ratio) of the salt by the addition of an effective quantity of a polyvalent alcohol that comprises 3 to 6 carbon atoms and 3 to 6 hydroxyl groups, preferably propylene glycol, sorbitol, and pentaerythritol. Preferred compositions are, for example, those that include, in wt % (USP): 18 to 45 wt % of an activated aluminum or aluminum-zirconium salt, 55 to 82 wt % of at least one anhydrous polyvalent alcohol having 3 to 6 carbon atoms and 3 to 6 hydroxyl groups, preferably propylene glycol, butylene glycol, diethylene glycol, dipropylene glycol, glycerol, sorbitol, and pentaerythritol, particularly preferably propylene glycol.

Also particularly preferred are complexes of activated perspiration-inhibiting aluminum or aluminum-zirconium salts with a polyvalent alcohol which include 20 to 50 wt %, particularly preferably 20 to 42 wt % activated perspiration-inhibiting aluminum or aluminum-zirconium salt and 2 to 16 wt % molecularly bound water, the remainder (to 100 wt %) being at least one polyvalent alcohol having 3 to 6 carbon atoms and 3 to 6 hydroxyl groups. Propylene glycol, propylene glycol/sorbitol mixtures, and propylene glycol/pentaerythritol mixtures are preferred alcohols of this kind. Complexes of this kind, preferred according to the present invention, of an activated perspiration-inhibiting aluminum or aluminum-zirconium salt with a polyvalent alcohol, are disclosed e.g. in U.S. Pat. No. 5,643,558 and U.S. Pat. No. 6,245,325.

Further preferred perspiration-inhibiting active substances are basic calcium-aluminum salts such as those disclosed e.g. in U.S. Pat. No. 2,571,030. These salts are manufactured by reacting calcium carbonate with aluminum chlorhydroxide or aluminum chloride and aluminum powder, or by adding calcium chloride dihydrate to aluminum chlorhydroxide.

Further preferred perspiration-inhibiting active substances are aluminum-zirconium complexes such as those disclosed e.g. in U.S. Pat. No. 4,017,599, which are buffered with salts of amino acids, in particular with alkali glycinate and alkaline-earth glycinates.

Further preferred perspiration-inhibiting active substances are activated aluminum or aluminum-zirconium salts such as those disclosed e.g. in U.S. Pat. No. 6,245,325 or U.S. Pat. No. 6,042,816, including 5 to 78 wt % (USP) of an activated perspiration-inhibiting aluminum or aluminum-zirconium salt, an amino acid or hydroxyalkane acid in a quantity such as to furnish a weight ratio of (amino acid or hydroxyalkane acid) to (Al+Zr) from 2:1 to 1:20, and preferably 1:1 to 1:10, as well as a water-soluble calcium salt in a quantity such as to furnish a Ca:(Al+Zr) weight ratio from 1:1 to 1:28, and preferably 1:2 to 1:25. Particularly preferred solid activated perspiration-inhibiting salt compositions, for example according to U.S. Pat. No. 6,245,325 or U.S. Pat. No. 6,042,816, include 48 to 78 wt % (USP), preferably 66 to 75 wt % of an activated aluminum or aluminum-zirconium salt, and 1 to 16 wt %, preferably 4 to 13 wt % molecularly bound water (water of hydration), furthermore a water-soluble calcium salt in a quantity sufficient that the Ca:(Al+Zr) weight ratio is 1:1 to 1:28, preferably 1:2 to 1:25, and sufficient amino acid that the (amino acid) to (Al+Zr) weight ratio is 2:1 to 1:20, preferably 1:1 to 1:10.

Further particularly preferred solid perspiration-inhibiting activated salt compositions, for example according to U.S. Pat. No. 6,245,325 or U.S. Pat. No. 6,042,816, include 48 to 78 wt % (USP), preferably 66 to 75 wt %, of an activated aluminum or aluminum-zirconium salt, and 1 to 16 wt %, preferably 4 to 13 wt % molecularly bound water (water of hydration), furthermore water-soluble calcium salt in a quantity sufficient that the Ca:(Al+Zr) weight ratio is 1:1 to 1:28, preferably 1:2 to 1:25, and sufficient glycine that the (glycine) to (Al+Zr) weight ratio is 2:1 to 1:20, preferably 1:1 to 1:10.

Further particularly preferred solid perspiration-inhibiting activated salt compositions, for example according to U.S. Pat. No. 6,245,325 or U.S. Pat. No. 6,042,816, include 48 to 78 wt % (USP), preferably 66 to 75 wt % of an activated aluminum or aluminum-zirconium salt, and 1 to 16 wt %, preferably 4 to 13 wt % molecularly bound water, furthermore water-soluble calcium salt in a quantity sufficient that the Ca:(Al+Zr) weight ratio is 1:1 to 1:28, preferably 1:2 to 1:25, and sufficient hydroxyalkane acid that the (hydroxyalkane acid) to (Al+Zr) weight ratio is 2:1 to 1:20, preferably 1:1 to 1:10.

Water-soluble calcium salts preferred for stabilization of the perspiration-inhibiting salts are selected from calcium chloride, calcium bromide, calcium nitrate, calcium citrate, calcium formate, calcium acetate, calcium gluconate, calcium ascorbate, calcium lactate, calcium glycinate, calcium carbonate, calcium sulfate, calcium hydroxide, and mixtures thereof.

Amino acids preferred for stabilization of the perspiration-inhibiting salts are selected from glycine, alanine, leucine, isoleucine, β-alanine, valine, cysteine, serine, tryptophan, phenylalanine, methionine, β-amino-n-butanoic acid, and γ-amino-n-butanoic acid and salts thereof, in each case in the d-form, the l-form, and the dl-form; glycine is particularly preferred.

Hydroxyalkane acids preferred for stabilization of the perspiration-inhibiting salts are selected from glycolic acid and lactic acid.

Further preferred perspiration-inhibiting active agents are activated aluminum or aluminum-zirconium salts such as those disclosed e.g. in U.S. Pat. No. 6,902,723, including 5 to 78 wt % (USP) of an activated perspiration-inhibiting aluminum or aluminum-zirconium salt, an amino acid or hydroxyalkane acid in a quantity such as to furnish a weight ratio of (amino acid or hydroxyalkane acid) to (Al+Zr) from 2:1 to 1:20 and preferably 1:1 to 1:10, as well as a water-soluble strontium salt in a quantity such as to furnish an Sr:(Al+Zr) weight ratio from 1:1 to 1:28, and preferably 1:2 to 1:25.

Particularly preferred solid activated perspiration-inhibiting salt compositions, for example according to U.S. Pat. No. 6,902,723, include 48 to 78 wt % (USP), preferably 66 to 75 wt % of an activated aluminum or aluminum-zirconium salt, and 1 to 16 wt %, preferably 4 to 13 wt % molecularly bound water, furthermore water-soluble strontium salt in a quantity sufficient that the Sr:(Al+Zr) weight ratio is 1:1 to 1:28, preferably 1:2 to 1:25, and sufficient amino acid that the (amino acid) to (Al+Zr) weight ratio is 2:1 to 1:20, preferably 1:1 to 1:10.

Further particularly preferred solid perspiration-inhibiting activated salt compositions, for example according to U.S. Pat. No. 6,902,723, include 48 to 78 wt % (USP), preferably 66 to 75 wt % of an activated aluminum or aluminum-zirconium salt, and 1 to 16 wt %, preferably 4 to 13 wt % molecularly bound water, furthermore water-soluble strontium salt in a quantity sufficient that the Sr:(Al+Zr) weight ratio is 1:1 to 1:28, preferably 1:2 to 1:25, and sufficient glycine that the (glycine) to (Al+Zr) weight ratio is 2:1 to 1:20, preferably 1:1 to 1:10.

Further particularly preferred solid perspiration-inhibiting activated salt compositions, for example according to U.S. Pat. No. 6,902,723, include 48 to 78 wt % (USP), preferably 66 to 75 wt % of an activated aluminum or aluminum-zirconium salt, and 1 to 16 wt %, preferably 4 to 13 wt % molecularly bound water, furthermore water-soluble strontium salt in a quantity sufficient that the Sr:(Al+Zr) weight ratio is 1:1 to 1:28, preferably 1:2 to 1:25, and sufficient hydroxyalkane acid that the (hydroxyalkane acid) to (Al+Zr) weight ratio is 2:1 to 1:20, preferably 1:1 to 1:10.

Further preferred activated aluminum salts are those of the general formula Al₂(OH)₆₋ₐXa, in which X is Cl, Br, I, or NO₃ and “a” is a value from 0.3 to 5, preferably from 0.8 to 2.5, and particularly preferably 1 to 2, so that the molar ratio Al:X is 0.9:1 to 2.1:1, as disclosed e.g. in U.S. Pat. No. 6,074,632. Some water of hydration is generally associatively bound into these salts, typically 1 to 6 mol water per mol salt. Aluminum chlorohydrate is particularly preferred (i.e. X is Cl in the formula above), and especially 5/6-basic aluminum chlorohydrate in which “a” is equal to 1, so that the molar ratio of aluminum to chlorine is 1.9:1 to 2.1:1.

Preferred activated aluminum-zirconium salts are those that represent mixtures or complexes of the above-described aluminum salts with zirconium salts of the formula ZrO(OH)_{2-pb}Y_{b}, where Y is Cl, Br, I, NO₃, or SO₄, b is a rational number from 0.8 to 2, and p is the valency of Y, as disclosed e.g. in U.S. Pat. No. 6,074,632. The zirconium salts as a rule likewise have some associatively bound water of hydration, typically 1 to 7 mol water per mol salt. The zirconium salt is preferably zirconyl hydroxychloride having the formula ZrO(OH)_{2-b-}Cl_{b}, in which b is a rational number from 0.8 to 2, preferably 1.0 to 1.9. Preferred aluminum-zirconium salts have an Al:Zr molar ratio from 2 to 10 and a metal:(X+Y) ratio from 0.73 to 2.1, preferably 0.9 to 1.5. A particularly preferred salt is aluminum zirconium chlorohydrate (i.e. X and Y are Cl), which has an Al:Zr ratio from 2 to 10 and a molar metal:Cl ratio from 0.9 to 2.1. The term “aluminum zirconium chlorohydrate” encompasses the tri-, tetra-, penta- and octachiorohydrate forms.

Further preferred perspiration-inhibiting active agents are disclosed in U.S. Pat. No. 6,663,854 and US 20040009133.

Preferred aluminum zirconium chlorohydrates generally have the empirical formula AlₙZr(OH)_{[3n+4-m(n+1)]}(Cl)_{[m(n+1)}], where n=2.0 to 10.0, preferably 3.0 to 8.0, m=0.77 to 1.11 (corresponding to a molar ratio of metal (Al+Zr) to chloride from 1.3 to 0.9), preferably m=0.91 to 1.11 (corresponding to M:Cl=1.1 to 0.9), and particularly preferably m=1.00 to 1.11 (corresponding to M:Cl=1.0 to 0.9), also very preferably m=1.02 to 1.11 (corresponding to M:Cl=0.98 to 0.9) and very preferably m=1.04 to 1.11 (corresponding to M:Cl=0.96 to 0.9).

Some water of hydration is generally associatively bound in these salts, typically 1 to 6 mol water per mol salt, corresponding 1 to 16 wt %, preferably 4 to 13 wt % water of hydration.

Preferred aluminum zirconium chlorohydrates are usually associated with an amino acid in order to prevent polymerization of the zirconium species during manufacture. Preferred stabilizing amino acids are selected from glycine, alanine, leucine, isoleucine, β-alanine, cysteine, valine, serine, tryptophan, phenylalanine, methionine, β-amino-n-butanoic acid, and γ-amino-n-butanoic acid, and salts thereof, respectively in the d-form, l-form, and dl-form; glycine is particularly preferred. The amino acid is included in the salt in a quantity from 1 to 3 mol, preferably 1.3 to 1.8 mol, in each case per mol of zirconium.

Preferred perspiration-inhibiting salts are aluminum zirconium tetrachlorohydrate (molar Al:Zr ratio=2 to 6; molar metal-to-chloride ratio M:Cl=0.9 to 1.3), in particular salts having a molar metal-to-chloride ratio (M:Cl) from 0.9 to 1.1, preferably 0.9 to 1.0.

Also preferred according to the present invention are aluminum zirconium chlorohydrate glycine salts that are stabilized with betaine ((CH₃)₃N⁺—CH₂—COO³¹ ). Particularly preferred corresponding compounds have a molar ratio of (total [betaine+glycine]) to Zr from (0.1 to 3.0):1, preferably (0.7 to 1.5):1, and a molar ratio of betaine to glycine of at least 0.001:1. Corresponding compounds are disclosed, for example, in U.S. Pat. No. 7,105,691.

In a particularly preferred embodiment according to the present invention, a so-called “activated” salt is included as a particularly effective antiperspirant salt, in particular one having a high HPLC peak 5 aluminum content, in particular having a peak 5 area of at least 33%, particularly preferably at least 45%, based on the total area under peaks 2 to 5, measured by HPLC in a 10 wt % aqueous solution of the active agent under conditions in which the aluminum species are resolved into at least four successive peaks (referred to as peaks 2 to 5). Preferred aluminum-zirconium salts having a high HPLC peak 5 aluminum content (also called “E⁵AZCH”) are disclosed, for example, in U.S. Pat. No. 6,436,381 and U.S. Pat. No. 6,649,152.

Also preferred are those activated “E⁵AZCH” salts whose HPLC peak 4 to peak 3 area ratio is equal to at least 0.4, preferably at least 0.7, particularly preferably at least 0.9.

Further particularly preferred antiperspirant active agents are those aluminum-zirconium salts having a high HPLC peak 5 aluminum content that are additionally stabilized with a water-soluble strontium salt and/or with a water-soluble calcium salt. Corresponding salts are disclosed, for example, in U.S. Pat. No. 6,923,952.

The antiperspirant active agents can be used as nonaqueous solutions or as a glycolic solubilizate. Preferably, however, the perspiration-inhibiting active agents are present in undissolved, suspended form.

If the perspiration-inhibiting active agents are present in undissolved form, suspended in a carrier not miscible with water, it is preferred for reasons of product stability that their particles have a number-average particle size from 0.1 to 200 μm, preferably 1 to 150 μm, particularly preferably 3 to 100 μm, and extraordinarily preferably 5 to 80 μm. Further preferred perspiration-inhibiting active agent particles have a volume-average particle size from 0.2 to 220 μm, preferably 3 to 160 μm, particularly preferably 4 to 125 μm, with further preference 5 to 120 μm, and extraordinarily preferably 10 to 80 μm.

Preferred perspiration-inhibiting aluminum-zirconium salts have a molar metal-to-chloride ratio from 0.9 to 1.5, preferably 0.9 to 1.3, particularly preferably 0.9 to 1.1.

Zirconium-free aluminum salts particularly preferred according to the present invention have a molar metal-to-chloride ratio from 1.9 to 2.1. Zirconium-free aluminum sesquichlorohydrates particularly preferred according to the present invention have a molar metal-to-chloride ratio from 1.5:1 to 1.8:1.

Particularly preferred compositions according to the present invention and used according to the present invention are characterized in that the at least one antiperspirant active agent is included in a quantity from 3 to 35 wt %, preferably 5 to 30 wt %, and particularly preferably 10 to 27 wt %, based on the total weight of active substance (USP), without water of crystallization, in the total composition.

In a particularly preferred embodiment, the composition according to the present invention or used according to the present invention includes an astringent aluminum salt, in particular aluminum chlorohydrate, particularly aluminum chlorohydrate having an active substance content (USP), without water of crystallization, from 72 to 88 wt %, based on the raw material as such. Preferred non-activated aluminum chlorohydrates are obtainable, for example, in powder form as Micro Dry® Ultrafine or Superultrafine from Reheis, Microdry 323 from Summit, as Chlorhydrol® and in activated form as Reach® 501 from Reheis. An aluminum sesquichlorohydrate that is likewise particularly preferred is offered under the name Reach® 301 from Summit (formerly Reheis). Also particularly preferred are activated aluminum chlorohydrates that are obtainable under the names Reach® 101 and Reach® 103, AACH-7171 from Summit (formerly Reheis).

The use of aluminum zirconium tetrachlorohydrex glycine complexes, which are marketed as a powder for example by Summit (formerly Reheis) under the name Rezal® 36 GP or AZG-364 or 369 by Summit, in activated quality as Reach® 908, can also be particularly preferred according to the present invention. Aluminum zirconium pentachlorohydrex glycine complexes (AAZG-3108 or AAZG-3110 of Summit) are also preferred perspiration-inhibiting active agents.

In a further preferred embodiment the compositions according to the present invention or used according to the present invention are introduced along with a propellant into an aerosol delivery apparatus.

Propellants (propellant gases) suitable according to the present invention are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluorethane, monochlorodifluoromethane, 1,1-difluoroethane, both individually and in combination. Hydrophilic propellant gases such as, for example, carbon dioxide can also be used advantageously for purposes of the present invention if the selected proportion of hydrophilic gases is low, and lipophilic propellant gas (e.g. propane/butane) is present in excess. Propane, n-butane, isobutane, and mixtures of these propellant gases are particularly preferred.

In a further particularly preferred embodiment the compositions according to the present invention or used according to the present invention include at least one propellant that is selected from at least one compound having 3 to 10 carbon atoms in accordance with formula (PROP-I)

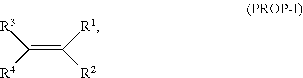

in which the residues R¹, R², R³, and R⁴ signify, mutually independently, a hydrogen atom, a bromine atom, a fluorine atom, or a (C₁ to C₆) alkyl group substituted with at least one fluorine atom,
or two of the residues R¹, R², R³, and R⁴ form a five- or six-membered ring, with the provision that at least one of the residues R¹, R², R³, or R⁴ denotes a hydrogen atom or a fluorine atom, and
at least one of the residues R¹, R², R³, or R⁴ denotes a (C₁ to C₆) alkyl group substituted with at least one fluorine atom, or at least two of the residues R¹, R², R³, and R⁴ form a five- or six-membered ring.

Fluorinated alkene propellants according to formula (PROP-I) have, as compared with non-fluorinated hydrocarbon propellants such as propane, propene, n-butane, isobutane, isobutene, n-pentane, pentene, isopentane, isopentene, methane, and ethane, a lower global warming potential and furthermore a relative low ground-level ozone formation potential (photochemical ozone creation potential=POCP) and a lower ozone depletion potential (=ODP) in the upper atmosphere, and are therefore more environmentally compatible. Their disadvantage at present is principally their low availability and very high cost.

Preferred propellants according to formula (PROP-I) are selected from at least one compound having 3 to 10 carbon atoms in accordance with the above formula (PROP-I) in which in which R¹, R², R³, and R⁴ mutually independently signify a hydrogen atom, a fluorine atom, or a (C₁ to C₆) alkyl group substituted with at least one fluorine atom, with the provision that at least one of the residues R¹, R², R³, or R⁴ denotes a hydrogen atom or a fluorine atom, and at least one of the residues R¹, R², R³, or R⁴ denotes a (C₁ to C₆) alkyl group substituted with at least one fluorine atom.

Particularly preferred propellants in accordance with formula (PROP-I) are selected from compounds of the formula E-R¹CH═CHR² or the formula Z—R¹CH═CHR², in which R¹ and R² mutually independently represent a perfluorinated C₁ to C₆ alkyl group.

Further preferred propellants in accordance with formula (PROP-I) are selected from CF₃CF═CHF, CF₃CH═CF₂, CHF₂CF═CF₂, CHF₂CH═CHF, CF₃CF═CH₂, CF₃CH═CHF, CH₂FCF═CF₂, CHF₂CH═CF₂, CHF₂CF═CHF, CHF₂CF═CH₂, CF₃CH═CH₂, CH₃CF═CF₂, CH₂FCH═CF₂, CH₂FCF═CHF, CHF₂CH═CHF, CF₃CF═CFCF₃, CF₃CF₂CF═CF₂, CF₃CF═CHCF₃, CF₃CF₂CF═CH₂, CF₃CH═CHF₃, CF₃CF₂CH═CH₂, CF₂═CHCF₂CF₃, CF₂═CHCF₂CF₃, CF₂═CFCHFCF₃, CF₂═CFCF₂CHF₂, CHF₂CH═CHCF₃, (CF₃)₂C═CHCF₃, CF₃CF═CHCF₂CF₃, CF₃CH═CFCF₂CF₃, (CF₃)₂CFCH═CH₂, CF₃CF₂CF₂CH═CH₂, CF₃(CF₂)₃CF═CF₂, CF₃CF₂CF═CFCF₂CF₃, (CF₃)₂C═C(CF₃)₂, (CF₃)₂CFCF═CHCF₃, CF₂═CFCF₂CH₂F, CF₂═CFCHFCHF₂, CH₂═C(CF₃)₂, CH₂CF₂CF═CF₂, CH₂FCF═CFCHF₂, CH₂FCF₂CF═CF₂, CF₂═C(CF₃)(CH₃), CH₂═C(CHF₂)(CF₃), CH₂═CHCF₂CHF₂, CF₂═C(CHF₂)(CH₃), CHF═C(CF₃)(CH₃), CH₂═C(CHF₂)₂, CF₃CF═CFCH₃, CH₃CF═CHCF₃, CF₂═CF(CF₂)₂CF₃, CHF═CF(CF₂)₂CF₃, CF₂═CH(CF₂)₂CF₃, CF₂═CF(CF₂)₂CHF₂, CHF₂CF═CFCF₂CF₃, CF₃CF═CFCF₂CHF₂, CF₃CF═CFCHFCF₃, CHF═CFCF(CF₃)₂, CF₂═CFCH(CF₃)₂, CF₃CH═C(CF₃)₂, CF₂═CHCF(CF₃)₂, CH₂═CF(CF₂)₂CF₃, CHF═CF(CF₂)₂CHF₂, CH₂═C(CF₃)C₂F₅, CF₂═CHCH(CF₃)₂, CHF═CHCF(CF₃)₂, CF₂═C(CF₃)CH₂CF₃, CH₂═CF(CF₂)₂CHF₂, CF₂═CHCF₂CH₂CF₃, CF₃CF═C(CF₃)CH₃, CH₂═CFCH(CF₃)₂, CHF═CHCH(CF₃)₂, CH₂FCH═C(CF₃)₂, CH₃ CF═C (CF₃)₂, CH₂═CHCF₂CHF CF₃, CH₂═C(CF₃)CH₂CF₃, (CF₃)₂C═CHC₂F₅, CH₂═CHC(CF₃)₃, (CF₃)₂C—C(CH₃)CF₃, CH₂—CFCF₂CH(CF₃)₂, CF₃CF═C(CH₃)C₂F₅, CF₃CH═CHCH(CF₃)₂, CH₂═CH(CF₂)₃CHF₂, (CF₃)₂C—CHCF₂CH₃, CH₂═C(CF₃)CH₂C₂F₅, CH₂═CHCH₂CF₂CF₂CF₃, C₂F₅CF═CFC₂H₅, CH₂═CHCH₂CF(CF₃)₂, CF₃CF═CHCH(CF₃)(CH₃), (CF₃)₂C═CFC₂H₅, cyclo-CF₂CF₂CF₂CH═CH—, cyclo-CF₂CF₂CH═CH—, CF₃CF₂CF₂C(CH₃)—CH₂, CF₃CF₂CF₂CH═CHCH₃, cyclo-CF₂CF₂CF═CF—, cyclo-CF₂CF═CFCF₂CF₂—, cyclo-CF₂CF═CFCF₂CF₂CF₂—, CF₃CF₂CF₂CF₂CH═CH₂, CF₃CH═CHC₂F₅, C₂F₅CH═CHC₂F₅, CF₃CH═CHCF₂CF₂CF₃, CF₃CF═CFC₂F₅, CF₃CF═CFCF₂CF₂CF₂CF₃, C₂F₅CF═CFCF₂CF₂CF₃, CF₃CH═CFCF₂CF₂CF₂CF₃, CF₃CF═CHCF₂CF₂CF₂CF₃, C₂F₅CH═CFCH₂CH₂CH₃, C₂F₅CF═CHCF₂CF₂CF₃,CF₃CF₂CF₂CF═CHCH₃, C₂F₅CF═CHCH₃, (CF₃)₂C═CHCH₃, CF₃C(CH₃)═CHCF₃, CHF═CFC₂F₅, CHF₂CF═CFCF₃, (CF₃)₂C═CHF, CH₂FCF═CFCF₃, CHF═CHC₂F₅, CHF₂CH═CFCF₃, CHF═CFCHFCF₃, CF₃CH═CFCHF₂, CHF═CFCF₂CHF₂, CHF₂CF═CFCHF₂, CH₂CF═CFCF₃, CH₂FCH═CFCF₃, CH₂═CFCHFCF₃, CH₂═CFCF₂CHF₂, CF₃CH═CFCH₂F, CHF═CFCH₂CF₃, CHF═CHCHFCF₃, CHF═CHCF₂CHF₂, CHF₂CF═CHCHF₂, CHF═CFCHFCHF₂, CF₃CF═CHCH₃, CF₂═CHCF₂Br, CHF═CBrCHF₂, CHBr═CHCF₃, CF₃CBr═CFCF₃, CH₂═CBrC₂F₅, CHBr═CHC₂F₅, CH₂═CH(CF₂)₂Br, CH₂═CHCBrFCF₃, CH₃CBr═CHCF₃, CF₃CBr═CHCH₃, (CF₃)₂C═CHBr, CF₃CF═CBrC₂F₅, E-CHF₂CBr═CFC₂F₅, Z—CHF₂CBr═CFC₂F₅, CF₂═CBrCHFC₂F₅, (CF₃)₂CFCBr═CH₂, CHBr═CF(CF₂)₂CHF₂, CH₂═CBrCF₂CF₂CF₃, CF₂═C(CH₂Br)CF₃, CH₂═C(CBrF₂)CF₃, (CF₃)₂CHCH═CHBr, (CF₃)₂C═CHCH₂Br, CH₂═CHCF(CF₃)CBrF₂, CF₂═CHCF₂CH₂CBrF₂, CFBr═CHCF₃, CFBr═CFCF₃, and CH₂═CBrCF₂CF₂CF₂CF₃, respectively in the E form or Z form, as well as mixtures of the aforesaid component.

Particularly preferred compositions according to the present invention or used according to the present invention include E-CF₃CH═CHF (E-1,3,3,3-tetrafluoroprop-1-ene) as a propellant of formula (PROP-I).

Further particularly preferred compositions according to the present invention or used according to the present invention are characterized in that non-fluorinated hydrocarbons having one to six carbon atoms are included in a total quantity from 0 to 50 wt %, preferably 0 to 30 wt %, particularly preferably 0 to 10 wt %, based in each case on the weight of all propellants included.

Extraordinarily preferred compositions according to the present invention or used according to the present invention are characterized in that the propellant comprises 0 wt % non-fluorinated hydrocarbons having one to six carbon atoms, as well as 40 to 100 wt %, preferably 70 to 99 wt %, extraordinarily preferably 80 to 93 wt % E-CF₃CH═CHF (E-1,3,3,3-tetrafluoroprop-1-ene), based in each case on the weight of all propellants included.

Compositions according to the present invention or used according to the present invention that include the propellant in a quantity from 10 to 90 wt %, preferably 40 to 85 wt %, and particularly preferably 50 to 80 wt %, based in each case on the total weight of the preparation made up of components a) to d) and the propellant, are preferred according to the present invention.

Suitable packages for propellant-including compositions according to the present invention or used according to the present invention—so-called compressed-gas containers or spray cans—are vessels made of metal (aluminum, tinplate, tin), of shielded or non-shattering plastic, or of glass that is coated externally with plastic, in the selection of which pressure resistance and resistance to breakage, corrosion resistance, and ease of filling, as well as aesthetic considerations, handling, imprintability, etc. play a role. Special internal protection lacquers ensure corrosion resistance with respect to the compositions according to the present invention.

Compositions preferred or preferably used according to the present invention that include an undissolved antiperspirant active agent, in particular an aluminum salt, suspended in an oil-including carrier, for example antiperspirant roll-ons and antiperspirant sprays sprayable using a propellant, include, for stable suspension of the undissolved constituents, a suspension agent that is selected from lipophilic thickening agents. Lipophilic thickening agents preferred according to the present invention are selected from hydrophobized clay minerals, in particular from hydrophobically modified hectorites and bentonites such as those obtainable, for example, under the INCI names Disteardimonium Hectorite, Stearalkonium Hectorite, Stearalkonium Bentonite, Quatemium-18 Hectorite, Quatemium-18 Bentonite, or Dihydrogenated Tallow Benzylmonium Hectorite. Compositions preferred or preferably used according to the present invention therefore include at least one hydrophobized clay mineral in a total quantity from 0.5 to 10 wt %, preferably 1 to 7 wt %, particularly preferably 2 to 6 wt %, extraordinarily preferably 3 to 5 wt %, based in each case on the total weight of the propellant-free composition.

Hydrophobized clay minerals of this kind usually require water, ethanol, or propylene carbonate as an activator, in a quantity from 0.3 to 3 wt %, preferably 0.5 to 2 wt %, based in each case on the total weight of the propellant-free composition according to the present invention.

Further lipophilic thickening agents preferred according to the present invention are selected from pyrogenic silicic acids, e.g. the commercial products of the Aerosil® series of Evonik Degussa. Hydrophobized pyrogenic silicic acids are particularly preferred; Silica Silylate and Silica Dimethyl Silylate are extraordinarily preferred.

Compositions preferred or preferably used according to the present invention are characterized in that they include at least one pyrogenic silicic acid, preferably at least one hydrophobized pyrogenic silicic acid, in a total quantity from 0.5 to 10 wt %, preferably 0.8 to 5 wt %, particularly preferably 1 to 4 wt %, extraordinarily preferably 1.5 to 2 wt %, based in each case on the total weight of the propellant-free composition according to the present invention.

Further compositions preferred or preferably used according to the present invention are characterized in that they include at least one hydrophobized pyrogenic silicic acid and at least one hydrophilic silicic acid.

Further compositions preferred or preferably used according to the present invention include, in addition to the active agents a) and b) according to the present invention, at least one further fragrance. The definition of a “fragrance” for purposes of the present Application corresponds to the definition usual in the art, as may be gathered from the RÖMPP Chemie Lexikon [Chemical Lexicon] as of December 2007. According to the latter, a “fragrance” is a chemical compound having an odor and/or taste that excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule. In order to distinguish volatile low-molecular-weight substances that are usually (and also for purposes of the present application) viewed and utilized not as fragrances but instead principally as solvents, for example ethanol, propanol, isopropanol, and acetone, from fragrances according to the present invention, fragrances according to the present invention have a molar mass from 74 to 300 g/mol, include at least one osmophoric group in the molecule, and have an odor and/or taste, i.e. they excite the receptors of the hair cells of the olfactory system.

Perfumes, perfume oils, perfume oil constituents, or individual fragrance compounds can be used to perfume the compositions according to the present invention or used according to the present invention. Perfume oils or fragrances can be, according to the present invention, individual fragrance compounds, e.g. synthetic products of the ester, ether, aldehyde, ketone, alcohol, and hydrocarbon types. Fragrance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmecyclate. Ethers include, for example, benzyl ethyl ether and ambroxan; aldehydes, for example, the linear alkanals having 8 to 18 carbon atoms, citral, citronellal, citronellyloxyacetaldehyde, cyclamenaldehyde, lilial, and bourgeonal; ketones, for example, the ionones, alpha-isomethylionone and methyl cedryl ketone; alcohols include citronellol, eugenol, geraniol, linalool, phenylethyl alcohol, alpha-terpineol, beta-terpineol, gamma-terpineol, and delta-terpineol; and hydrocarbons include principally terpenes such as limonene and pinene. Preferably, however, mixtures of different fragrances that together produce an attractive fragrance note are used.

Perfume oils of this kind can also include natural fragrance mixtures such as those accessible from plant sources, for example pine, citrus, jasmine, patchouli, rose, or ylang-ylang oil. Also suitable are muscatel sage oil, chamomile oil, clove oil, lemon balm oil, cinnamon leaf oil, linden blossom oil, juniper berry oil, vetiver oil, olibanum oil, galbanum oil, and labdanum oil, as well as orange blossom oil, neroli oil, orange peel oil, and sandalwood oil.

In order to be perceptible, a fragrance must be volatile; in addition to the nature of the functional groups and the structure of the chemical compound, the molecular weight also plays an important part. Most fragrances thus possess molar weights of up to approximately 200 dalton, while molar weights of 300 dalton and above represent something of an exception. Because of the differing volatility of fragrances, the odor of a perfume or fragrance made up of multiple fragrances changes during volatilization, the odor impressions being subdivided into a “top note,” “middle note” or “body,” and “end note” or “dry out.” Because the perception of an odor also depends a great deal on the odor intensity, the top note of a perfume or fragrance is not made up only of highly volatile compounds, while the end note comprises for the most part less-volatile, i.e. adherent fragrances. In the compounding of perfumes, more-volatile fragrances can, for example, be bound to specific fixatives, thereby preventing them from volatilizing too quickly. The division below of fragrances into “more-volatile” and “adherent” fragrances therefore makes no statement with regard to the odor impression and as to whether the corresponding fragrance is perceived as a top or middle note.

Adherent fragrances that are usable in the context of the present invention are, for example, the essential oils such as angelica oil, anise oil, arnica flower oil, basil oil, bay oil, bergamot oil, champaca flower oil, silver fir oil, silver fir cone oil, elemi oil, fennel oil, fir needle oil, galbanum oil, geranium oil, gingergrass oil, guaiac wood oil, balsam gurjun oil, helichrysum oil, ho oil, ginger oil, iris oil, cajeput oil, calamus oil, chamomile oil, camphor oil, kanaga oil, cardamom oil, cassia oil, pine needle oil, balsam copaiva oil, coriander oil, spearmint oil, caraway oil, cumin oil, lavender oil, lemon grass oil, lime oil, tangerine oil, lemon balm oil, ambrette seed oil, myrrh oil, clove oil, neroli oil, niaouli oil, olibanum oil, orange oil, oregano oil, palmarosa oil, patchouli oil, balsam peru oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, citron oil, and cypress oil. The higher-boiling or solid fragrances of natural or synthetic origin can, however, also be used advantageously in the context of the present invention as adherent fragrances or fragrance mixtures. Included among these compounds are the compounds recited below as well as mixtures thereof: ambrettolide, allyl acetate, alpha-amylcinnamaldehyde, anisealdehyde, anise alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bomyl acetate, alpha-bromostyrene, n-decylaldehyde, n-dodecylaldehyde, eugenol, eugenol methyl ether, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvacrol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl beta-naphthyl ketone, methyl-n-nonylacetaldehyde, methyl n-nonyl ketone, muscone, beta-naphthol ethyl ether, beta-naphthol methyl ether, nerol, nitrobenzene, n-nonylaldehyde, nonyl alcohol, n-octylaldehyde, p-oxyacetophenone, pentadecanolide, beta-phenylethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, alpha-terpineol, beta-terpineol, gamma-terpineol, delta-terpineol, thymene, thymol, gamma-undelactone, vanillin, veratrumaldehyde, cinnamaldehyde, cinnamyl alcohol, cinnamic acid, cinnamic acid ethyl ester, cinnamic acid benzyl ester.

Included among the more-volatile fragrances that can be used are, in particular, the lower-boiling fragrances of natural or synthetic origin, which can be used alone or in mixtures. Examples of more-volatile fragrances are alkyl isothiocyanates (alkyl mustard oils), butanedione, limonene, linalool, linalyl acetate and linalyl propionate, menthol, menthone, methyl-n-heptenone, phellandrene, phenylacetaldehyde, terpinyl acetate, citral, citronellal.

Compositions preferred or preferably used according to the present invention include, in addition to the active agents a) and b) according to the present invention, at least one further fragrance in a total quantity from 0.00001 to 10 wt %, preferably 0.5 to 7 wt %, particularly preferably 1 to 5 wt %, wherein the “wt %” indications refer to the total weight of the composition without taking into account any propellant that may be present.

A further subject of the present application is the use of a cosmetic composition including components a) to c),

a) at least one alkoxybenzene compound having the structural formula (I)

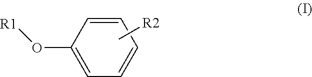

having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %,

b) at least one compound selected from cyclic monoterpene epoxies in a total quantity from 0.01 to 1 wt %, and menthol in a total quantity from 0.09 to 5 wt %, and from mixtures of said components,

c) a cosmetically acceptable carrier including at least one component selected from water, ethanol, an oil, and talc, as well as mixtures thereof, as well as optionally further carrier substances, adjuvants, and active agents,

to reduce body odor of the armpits and/or feet, wherein the “wt %” indications refer to the total weight of the composition without taking into account any propellant that may be present.

The statements made regarding the compositions according to the present invention apply mutatis mutandis with respect to further preferred embodiments of the uses according to the present invention, provided no reference is made to the water content of the compositions.

A further subject of the present application is a non-therapeutic, cosmetic method for reducing body odor, in which a cosmetic composition including components a) to c),

a) at least one alkoxybenzene compound having the structural formula (I)

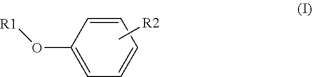

having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %,

b) at least one compound selected from cyclic monoterpene epoxies in a total quantity from 0.01 to 1 wt %, and menthol in a total quantity from 0.09 to 5 wt %, and from mixtures of said components,

c) a cosmetically acceptable carrier including at least one component selected from water, ethanol, an oil, and talc, as well as mixtures thereof, as well as optionally further carrier substances, adjuvants, and active agents,

is applied in an effective quantity onto the skin of the armpits and/or of the feet, wherein the “wt %” indications refer to the total weight of the composition without taking into account any propellant that may be present.

The statements made regarding the compositions according to the present invention apply mutatis mutandis with respect to further preferred embodiments of the method according to the present invention, provided no reference is made to the water content of the compositions.

### EXPERIMENTAL INVESTIGATIONS

An artificial perspiration mixture was produced by mixing different short-chain fatty acids at specific quantitative ratios (C6, C8, C9, C10, isovaleric acid). respective 0.5-wt % solutions in ethanol were produced of each of the active agents and active-agent combinations to be tested, applied in a standardized quantity onto filter paper, and dried for one hour at room temperature. The artificial perspiration mixture was then applied in a standardized quantity, and the filters were stored for 24 hours at room temperature in a closed container.

An odor evaluation of the samples was carried out by six trained testers using a scale from 0 (no difference from ethanol control) to 4 (very much better odor reduction as compared with ethanol control).

The results are depicted in Illustration 1.

A 0.5-wt % solution of anethole (raw material having a maximum of 0.5 wt % cis-anethole, based on the weight of total anethole) in ethanol proved to be particularly effective, with a reduction in perspiration odor of 2.42 units as compared with the ethanol control. Anethole is, however, characterized by an intense anise odor, so that in larger quantities it is not suitable for most commercial deodorants, since a strong anise odor is not accepted by consumers for a cosmetic product.

DL-Menthol and eucalyptol at a concentration of 0.5 wt % in ethanol reduced the unpleasant odor by 1.67 and 0.92 respectively, as compared with ethanol alone as a control.

For these individual substances as well, it is desirable not to use them alone at high concentration, so that their inherent odor does not undesirably dominate the odor of the overall composition.

A very good reduction in body odor was likewise obtained with the double combinations DL-menthol/eucalyptol, DL-menthol/anethole, and eucalyptol/anethole, an excessively strong inherent odor of the respective active-agent combinations being avoided.

A synergistic effect in reducing body odor was achieved for the triple combinations of anethole/eucalyptol/DL-menthol, since the expected value for the odor reduction, namely (1.67+0.92+2.42) units/3=1.67 units, was greatly exceeded by the experimentally determined reduction in body odor of 2.33 units.

The active-agent combinations according to the present invention, of alkoxybenzene compounds having the structural formula (I) and a cyclic monoterpene epoxy and/or menthol, exhibited an optimal masking effect with respect to perspiration odor and were notable for a very pleasant inherent odor that can be used for many deodorant compositions.

The improved protection from body odor resulting from the active-agent combinations according to the present invention was confirmed with statistical significance by a two-week utilization test using 200 test subjects.

The examples below are intended to elucidate the invention without limiting it thereto.

### EXAMPLES OF COMPOSITIONS ACCORDING TO THE PRESENT INVENTION

Unless otherwise indicated, all quantity indications are in wt %. “Propylene carbonate” means 4-methyl-1,3-dioxolan-2-one.

1. Perspiration Inhibiting Suspension Sprays

| | 1.1 | 1.2 |
|---|---|---|
| n-Butane | 72.50 | 72.70 |
| Propane | 12.00 | 12.00 |
| Isobutane | 1.50 | 1.50 |
| Cyclopentasiloxane | 6.00 | 6.00 |
| Aluminum chlorohydrate | 5.00 | 5.00 |
| Isopropyl myristate | 1.00 | 1.00 |
| Disteardimonium Hectorite | 0.38 | 0.38 |
| DL-menthol | 0.17 | 0.25 |
| Eucalyptol | 0.17 | 0.03 |
| Anethole | 0.17 | 0.05 |
| Propylene carbonate | 0.13 | 0.13 |
| Perfume | 1.00 | 1.00 |

2. Deodorant Spray

| | |
|---|---|
| n-Butane | 63.7 |
| Ethanol | 22.0 |
| Propane | 10.0 |
| Triethyl citrate | 1.5 |
| Isobutane | 1.3 |
| DL-menthol | 0.2 |
| Eucalyptol | 0.2 |
| Anethole | 0.2 |
| Phenoxyethanol | 0.2 |
| Vitamin E acetate | 0.1 |
| Perfume | 0.6 |

3. Perspiration-Inhibiting Roll-On

| | % |
|---|---|
| Water | 74.0 |
| Aluminum chlorohydrate | 20.0 |
| Steareth-21 | 1.5 |
| Steareth-2 | 2.4 |
| PPG-15 Stearyl Ether | 0.5 |
| DL-menthol | 0.2 |
| Eucalyptol | 0.2 |
| Anethole | 0.2 |
| Perfume | 1.0 |

4. Perspiration-Inhibiting Stick

| | |
|---|---|
| Cyclopentasiloxane | 35.2 |
| Stearyl alcohol | 24.0 |
| Aluminum Zirconium | 22.0 |
| Pentachlorohydrex GLY | |
| PPG-14 Butyl Ether | 10.0 |
| Hardened castor oil (e.g. Cutina HR) | 3.0 |
| Myristyl myristate | 1.5 |
| DL-menthol | 0.2 |
| Eucalyptol | 0.2 |
| Anethole | 0.2 |
| Silica Dimethyl Silylate | 1.4 |
| Silica | 0.3 |
| Perfume | 2.0 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for use as a deodorant, including components a) to d),
a) at least one alkoxybenzene compound having the structural formula (I) 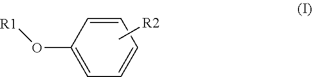
having the residues R¹ and R², wherein R¹ is selected from a C₁ to C₈ alkyl group and R² is selected from a C₁ to C₈ alkyl group and a C₂ to C₈ alkenyl group, in a total quantity from 0.01 to 1 wt %,
b) at least one compound selected from cyclic monoterpene epoxies in a total quantity from 0.01 to 1 wt % and menthol in a total quantity from 0.09 to 5 wt %, and from mixtures of said components,
c) 0 to 7 wt % water,
d) a cosmetically acceptable carrier including at least one component selected from ethanol, a cosmetic oil liquid under standard conditions, and talc, as well as mixtures thereof, as well as optionally further carrier substances, adjuvants, and active agents,
wherein the “wt %” indications refer to the total weight of the composition without taking into account any propellant that may be present.

2. The composition according to claim 1, wherein the at least one compound having the structural formula (I) is selected from compounds in which R¹ represents a methyl group or an ethyl group.

3. The composition according to claim 1, wherein the at least one compound having the structural formula (I) is selected from compounds in which R² is selected from an ethyl group, an n-propyl group, a 1-methylethyl group, an n-butyl group, a 1-propenyl group, and a 2-propenyl group.

4. The composition according to claim 1, wherein the at least one compound having the structural formula (I) is selected from compounds in which R¹ is a methyl group and R² is a 1-propenyl group, preferably selected from trans-anethole.

5. The composition according to claim 1, wherein the at least one cyclic monoterpene epoxy is selected from eucalyptol (=1,8-cineol, 1,8-epoxy-p-menthane, 1,3,3-trimethyl-2-oxabicyclo [2.2.2] octane) and 1,4-cineol 1-methyl-4-(1-methylethyl)-7-oxabicyclo [2.2.1]heptane, 1,4-epoxy-p-menthane), and mixtures thereof.

6. The composition according to claim 1, wherein the menthol is selected from L-menthol, D-menthol, and DL-menthol, preferably selected from DL-menthol.

7. The composition according to claim 1, wherein a) 0.01 to 1 wt % trans-anethole and b) 0.09 to 5 wt % menthol, preferably 0.09 to 5 wt % DL-menthol, are included, wherein the “wt %” indications refer to the total weight of the composition without taking into account any propellant that may be present.

8. The composition according to claim 1, wherein a) 0.01 to 1 wt % trans-anethole and b) 0.01 to 1 wt % eucalyptol (=1,8-cineol) are included, wherein the “wt %” indications refer to the total weight of the composition without taking into account any propellant that may be present.

9. The composition according to claim 1, wherein a) 0.01 to 1 wt % trans-anethole and b)i 0.01 to 1 wt % eucalyptol 1,8-cineol) and b)ii 0.09 to 5 wt % menthol are included, wherein the “wt %” indications refer to the total weight of the composition without taking into account any propellant that may be present.

10. The composition according to claim 4, wherein the weight ratio of all alkoxybenzene compounds having the structural formula (I) to cyclic monoterpene epoxies is in the range from 1:0.2 to 1:1.1.

11. The composition according to claim 1, wherein 0.01 to 1 wt % DL-menthol are included, wherein the “wt %” indications refer to the total weight of the composition without taking into account any propellant that may be present, and wherein the weight ratio of trans-anethole to menthol is in the range from 1:0.8 to 1:10.

12. The composition according to claim 1, wherein 0.01 to 1 wt % DL-menthol are included, wherein the “wt %” indications refer to the total weight of the composition without taking into account any propellant that may be present, and wherein the weight ratio of trans-anethole to eucalyptol is in the range from 1:0.2 to 1:1.1.

13. The composition according to claim 1, wherein 0.01 to 1 wt % DL-menthol are included, wherein the “wt %” indications refer to the total weight of the composition without taking into account any propellant that may be present, and wherein the weight ratio of menthol to eucalyptol is in the range from 1:1 to 20:1.

14. The composition according to claim 1, characterized in that 0.01 to 1 wt % DL-menthol are included, wherein the “wt %” indications refer to the total weight of the composition without taking into account any propellant that may be present, and wherein the weight ratio of trans-anethole to the sum of eucalyptol and menthol is [symbol]
